(19) United States
(12) Patent Application Publication
(10) Pub. No.: US 2024/0300911 A1
(43) Pub. Date: Sep. 12, 2024

(54) PURIFICATION METHOD FOR CITALOPRAM OR S-CITALOPRAM

(71) Applicants: ZHEJIANG HUAHAI PHARMACEUTICAL CO., LTD., Linhai Taizhou (CN); Zhejiang Huahai LiCheng Pharmaceutical Co., Ltd., Linhai Taizhou (CN)

(72) Inventors: Jian ZHANG, Linhai Taizhou (CN); Jichao WANG, Linhai Taizhou (CN); Zheng HAN, Linhai Taizhou (CN); Yihong XU, Linhai Taizhou (CN); Jiaxing HU, Linhai Taizhou (CN); Wenfeng HUANG, Linhai Taizhou (CN); Yongjun JIN, Linhai Taizhou (CN)

(73) Assignees: ZHEJIANG HUAHAI PHARMACEUTICAL CO., LTD., Linhai Taizhou (CN); Zhejiang Huahai LiCheng Pharmaceutical Co., Ltd., Linhai Taizhou (CN)

(21) Appl. No.: 18/261,480

(22) PCT Filed: Jan. 10, 2022
(86) PCT No.: PCT/CN2022/070997

§ 371 Date: Jul. 13, 2023

(30) Foreign Application Priority Data
Jan. 14, 2021 (CN) 202110047911.X

(51) Int. Cl. C07D 307/81

(52) U.S. Cl. CPC C07D 307/81

(57) ABSTRACT

The present invention provides a purification method for citalopram or S-citalopram or salts thereof, comprising the following steps: using a washing solution to treat a solution consisting of citalopram and a water-immiscible organic solvent and carrying out separation to obtain an organic layer containing citalopram, or using a washing solution to treat a solution consisting of S-citalopram and a water-immiscible organic solvent and carrying out separation to obtain an organic layer containing S-citalopram; and then, taking the organic layer and carrying out further separation to obtain citalopram or S-citalopram, or adding acid to form a salt and carrying out further separation to obtain an acid salt of citalopram or S-citalopram. The purification method provided by the present invention is simple in operation and high in impurity removal rate.

The present invention claims priority of Chinese Patent Application No. 202110047911.X, filed before the CNIPA on Jan. 14, 2021, titled “PURIFICATION METHOD FOR CITALOPRAM OR S-CITALOPRAM”, which is incorporated herein by reference in its entirety.

### FIELD OF THE INVENTION

The present invention relates to the technical field of pharmaceutical chemistry, in particular to a purification method for citalopram or s-citalopram.

### BACKGROUND OF THE INVENTION

Citalopram hydrobromide is a racemic mixture, wherein the active ingredient is mainly its levoisomer.

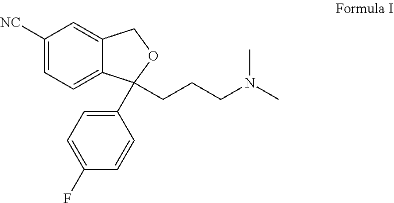 [SMILES: CN(C)CCCC1(c2ccc(F)cc2)OCc2cc(C#N)ccc21]

1-(3-Dimethylaminopropyl)-1-(4-fluorophenyl)-1,3-dihydroisobenzofuran-5-formaldehyde (Formula II), commonly known as aldehyde impurity, is an inherent impurity in citalopram hydrobromide and escitalopram (S-citalopram) oxalate. This impurity is similar in nature to the product and is extremely difficult to remove, and generally has a content level of about 0.1% to about 0.4%. Currently, there is no relevant literature reporting the source and removal of this impurity.

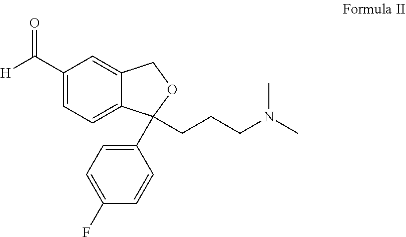 [SMILES: CN(C)CCCC1(c2ccc(F)cc2)OCc2cc(C=O)ccc21]

In view of the above problems regarding the impurity, it is necessary to develop a purification method for citalopram or S-citalopram.

### SUMMARY

The present invention provides a purification method for citalopram or S-citalopram or salts thereof, comprising the following steps:
(a) treating a solution consisting of citalopram and a water-immiscible organic solvent with a washing solution, and carrying out a separation to obtain an organic layer containing citalopram; or, treating a solution consisting of S-citalopram and a water-immiscible organic solvent with a washing solution, and carrying out a separation to obtain an organic layer containing S-citalopram; and
(b) further separating the organic layer obtained in step (a) to obtain citalopram or S-citalopram; or further adding an acid to form a salt and carrying out a separation to obtain an acid salt of citalopram or an acid salt of S-citalopram;
wherein, the washing solution as defined in step (a) is an aqueous solution comprising a washing agent, and the washing agent is selected from sulfite and thiosulfate.

In a preferred embodiment of the present invention:

the water-immiscible organic solvent as defined in step (a) is selected from the group consisting of toluene, ethyl acetate, methyl isobutyl ketone, and chlorobenzene, or any combinations thereof.

The solution consisting of citalopram and the water-immiscible organic solvent or the solution consisting of S-citalopram and the water-immiscible organic solvent as defined in step (a) is obtained by forming a free base from an acid salt of citalopram or acid salt of S-citalopram, which is obtained by the following process: mixing the acid salt of citalopram or the acid salt of S-citalopram, water and the water-immiscible organic solvent, adding a base to obtain a free base, and carrying out a separation to obtain an organic layer; or the solution can also be derived from a reaction solution in a synthesis process of citalopram or S-citalopram, and the reaction solution can be prepared with reference to technical solutions in the prior art. Preferably, the solution is obtained by forming a free base from citalopram hydrobromide or S-citalopram oxalate.

The washing agent in the washing solution as defined in step (a) is selected from the group consisting of sodium dithionite, sodium thiosulfate, and sodium sulfite, preferably is sodium dithionite.

A molar ratio of the washing agent and citalopram or S-citalopram ranges from 0.05 to 1.5.

A mass ratio of the washing agent and water in the washing solution as defined in step (a) ranges from 1% to 30%.

In one embodiment, an inorganic base is added to the washing solution as defined in step (a), and the inorganic base is selected from the group consisting of sodium carbonate, sodium bicarbonate, potassium carbonate, potassium bicarbonate, sodium hydroxide and potassium hydroxide, or any combinations thereof; preferably the inorganic base is sodium bicarbonate.

A mass ratio of the inorganic base and the washing agent ranges from 5% to 30%.

The acid as defined in step (b) is selected from the group consisting of hydrochloric acid, hydrobromic acid, phosphoric acid, oxalic acid, fumaric acid, acetic acid, p-toluenesulfonic acid and methanesulfonic acid; preferably the acid is hydrobromic acid or oxalic acid.

In the present invention, the washing solution as defined in step (a) is an aqueous solution containing washing agent. The preparation process is not limited in the present application, as long as it can achieve the purpose of the present invention. Exemplarily, it is prepared in accordance with the following steps: adding the washing agent and water, and optionally the inorganic base, to a container successively, and stirring the mixture until dissolved and clarified to obtain the washing solution.

The process of stirring the washing solution as defined in step (a) is not limited in the present invention, as long as it can achieve the purpose of the present invention. Exemplarily, it can be achieved by stirring the mixture at a temperature of 40° C. to 45° C. for 60 min to 90 min.

The process of further separating the organic layer to obtain citalopram or S-citalopram as defined in step (b) is not limited in the present invention, as long as it can achieve the purpose of the invention. Exemplarily, the separation is performed by concentrating the organic layer to dryness under reduced pressure.

In the present invention, the process of further adding acid to form a salt as defined in step (b) is performed by dissolving the organic layer that has been concentrated to dryness in an organic solvent under a controlled temperature. In the present invention, the organic solvent is selected from the group consisting of toluene, ethyl acetate, methanol, methyl isobutyl ketone and anhydrous ethanol, or any combinations thereof; and the temperature is controlled in the range of 40° C. to 60° C. Exemplarily, the organic layer is concentrated to dryness under reduced pressure, added to ethyl acetate and dissolved, and the temperature is controlled at 50° C. to 60° C.

The process of further adding acid to form a salt as defined in step (b) is performed by dissolving the organic layer and adding an acid dropwise to adjust pH=4±0.5 so as form the salt, and the acid salt of citalopram or acid salt of S-citalopram is obtained through separation. In the present invention, the acid is selected from the group consisting of hydrochloric acid, hydrobromic acid, phosphoric acid, oxalic acid, fumaric acid, acetic acid, p-toluenesulfonic acid, and methanesulfonic acid. Exemplarily, hydrobromic acid is added dropwise to adjust pH=4±0.5 to form a salt, and then the resultant is refluxed for 2 hours, cooled down to crystallize a crystal, which is then filtered and dried to obtain citalopram hydrobromide.

The purification method for citalopram or S-citalopram provided by the present invention is simple in operation and high in impurity removal rate, cheap and easily available in raw and auxiliary materials, and mild in conditions, which is conducive to the simple and efficient purification of citalopram or S-citalopram.

### DETAILED DESCRIPTION

The invention is further described in detail by specific examples below. All raw materials used in the examples are commercially available.

### Example 1

In a beaker, 2.5 g of sodium dithionite and 50 mL of drinking water were added successively, and the mixture was stirred until dissolved and clarified to obtain a washing solution.

In a three-necked flask, 25 g of citalopram hydrobromide (containing 0.364% aldehyde impurity), 200 mL of toluene and 100 mL of drinking water were added successively. The temperature was raised to 45° C. Ion-exchange membrane liquid caustic soda was slowly added dropwise to adjust pH of the aqueous solution to 12. The resultant was stirred until dissolved and clarified, and allowed to stand and layer. An organic layer was obtained by separation, and was added with the washing solution. Under a controlled temperature of 40° C. to 45° C., the mixture was stirred for 60 min and allowed to stand and layer. An organic layer was obtained by separation, and was concentrated to dryness under reduced pressure, which was then dissolved by 120 mL of ethyl acetate. Under a controlled temperature of 50° C. to 60° C., hydrobromic acid was added dropwise to adjust pH=4±0.5 to form a salt, and the resultant was then refluxed for 2 hours and cooled down to crystallize a crystal, which was then filtered and dried to obtain citalopram hydrobromide. Yield: 90%; purity: 99.7%; aldehyde impurity: 0.04%; impurity removal rate: 89.0%.

### Example 2

In a beaker, 2.5 g of sodium dithionite, 50 mL of drinking water and 0.25 g of sodium bicarbonate were added successively, and the mixture was stirred until dissolved and clarified to obtain a washing solution.

In a three-necked flask, 25 g of citalopram hydrobromide (containing 0.470% aldehyde impurity), 200 mL of toluene and 100 mL of drinking water were added successively. The temperature was raised to 45° C. Ion-exchange membrane liquid caustic soda was slowly added dropwise to adjust pH of the aqueous solution to 12. The resultant was stirred until dissolved and clarified, and allowed to stand and layer. An organic layer was obtained by separation, and was added with the washing solution. Under a controlled temperature of 40° C. to 45° C., the mixture was stirred for 60 min and allowed to stand and layer. An organic layer was obtained by separation, and was concentrated to dryness under reduced pressure, which was then dissolved by 120 mL of ethyl acetate. Under a controlled temperature of 50° C. to 60° C., hydrobromic acid was added dropwise to adjust pH=4±0.5 to form a salt, and the resultant was then refluxed for 2 hours and cooled down to crystallize a crystal, which was then filtered and dried to obtain citalopram hydrobromide. Yield: 91%; purity: 99.8%; aldehyde impurity: 0.029%; impurity removal rate: 93.8%.

### Example 3

In a beaker, 0.5 g of sodium dithionite, 50 mL of drinking water and 0.05 g of sodium bicarbonate were added successively, and the mixture was stirred until dissolved and clarified to obtain a washing solution.

In a three-necked flask, 25 g of S-citalopram oxalate (containing 0.364% aldehyde impurity), 200 mL of methyl isobutyl ketone and 100 mL of drinking water were added successively. The temperature was raised to 45° C. 30% potassium hydroxide solution was slowly added dropwise to adjust pH of the aqueous solution to 12. The resultant was stirred until dissolved and clarified, and allowed to stand and layer. An organic layer was obtained by separation, and was added with the washing solution. Under a controlled temperature of 40° C. to 45° C., the mixture was stirred for 60 min and allowed to stand and layer. An organic layer was obtained by separation, and was concentrated to dryness under reduced pressure, which was then added with 48 mL of anhydrous ethanol. The resultant was stirred until dissolved and clarified, and under a controlled temperature of 40° C. to 50° C. 9.1 g of oxalic acid was added. The mixture was stirred for 2 hours and cooled down to crystallize a crystal, which was then filtered and dried to obtain S-citalopram oxalate. Yield: 88%; purity: 99.9%; aldehyde impurity: 0.033%; impurity removal rate: 90.9%.

### Example 4

In a beaker, 2.3 g of sodium thiosulfate, 50 mL of drinking water and 0.23 g of sodium carbonate were added successively, and the mixture was stirred until dissolved and clarified to obtain a washing solution.

In a three-necked flask, 25 g of citalopram hydrobromide (containing 0.470% aldehyde impurity), 200 mL of ethyl acetate and 100 mL of drinking water were added successively. The temperature was raised to 45° C. Ion-exchange membrane liquid caustic soda was slowly added dropwise to adjust pH of the aqueous solution to 12. The resultant was stirred until dissolved and clarified, and allowed to stand and layer. An organic layer was obtained by separation, and was added with the washing solution. Under a controlled temperature of 40° C. to 45° C., the mixture was stirred for 60 min and allowed to stand and layer. An organic layer was obtained by separation, and was concentrated to dryness under reduced pressure, which was then dissolved by 120 mL of ethyl acetate. Under a controlled temperature of 50° C. to 60° C., hydrobromic acid was added dropwise to adjust pH=4±0.5 to form a salt, and the resultant was refluxed for 2 hours and cooled down to crystallize a crystal, which was then filtered and dried to obtain citalopram hydrobromide. Yield: 92%; purity: 99.8%; aldehyde impurity: 0.06%; impurity removal rate: 87.2%.

### Example 5

In a beaker, 1.8 g of sodium sulfite, 50 mL of drinking water and 0.54 g of potassium bicarbonate were added successively, and the mixture was stirred until dissolved and clarified to obtain a washing solution.

In a three-necked flask, 25 g of citalopram hydrobromide (containing 0.303% aldehyde impurity), 200 mL of chlorobenzene and 100 mL of drinking water were added successively. The temperature was raised to 45° C. Ion-exchange membrane liquid caustic soda was slowly added dropwise to adjust pH of the aqueous solution to 12. The resultant was stirred until dissolved and clarified, and allowed to stand and layer. An organic layer was obtained by separation, and was added with the washing solution. Under a controlled temperature of 40° C. to 45° C., the mixture was stirred for 60 min and allowed to stand and layer. An organic layer was obtained by separation, and was concentrated to dryness under reduced pressure, which was then dissolved by 120 mL of ethyl acetate. Under a controlled temperature of 50° C. to 60° C., hydrobromic acid was added dropwise to adjust pH=4±0.5 to form a salt, and the resultant was refluxed for 2 hours and cooled down to crystallize a crystal, which was then filtered and dried to obtain citalopram hydrobromide. Yield: 91%; purity: 99.7%; aldehyde impurity: 0.023%; impurity removal rate: 92.4%.

### Example 6

In a beaker, 15 g of sodium dithionite, 50 mL of drinking water and 0.75 g of potassium carbonate were added successively, and the mixture was stirred until dissolved and clarified to obtain a washing solution.

In a three-necked flask, 25 g of citalopram hydrobromide (containing 0.303% aldehyde impurity), 200 mL of toluene and 100 mL of drinking water were added successively. The temperature was raised to 45° C. Ion-exchange membrane liquid caustic soda was slowly added dropwise to adjust pH of the aqueous solution to 12. The resultant was stirred until dissolved and clarified, and allowed to stand and layer. An organic layer was obtained by separation, and was added with the washing solution. Under a controlled temperature of 40° C. to 45° C., the mixture was stirred for 60 min and allowed to stand and layer. An organic layer was obtained by separation, and was concentrated to dryness under reduced pressure, which was then dissolved by 120 mL of ethyl acetate. Under a controlled temperature of 50° C. to 60° C., hydrobromic acid was added dropwise to adjust pH=4±0.5 to form a salt, and the resultant was refluxed for 2 hours and cooled down to crystallize a crystal, which was then filtered and dried to obtain citalopram hydrobromide. Yield: 90%; purity: 99.8%; aldehyde impurity: 0.041%; impurity removal rate: 86.5%.

### Example 7

In a beaker, 2.5 g of sodium dithionite, 50 mL of drinking water and 0.25 g of sodium hydroxide were added successively, and the mixture was stirred until dissolved and clarified to obtain a washing solution.

In a three-necked flask, 25 g of citalopram hydrobromide (containing 0.522% aldehyde impurity), 200 mL of methyl isobutyl ketone and 100 mL of drinking water were added successively. The temperature was raised to 45° C. Ion-exchange membrane liquid caustic soda was slowly added dropwise to adjust pH of the aqueous solution to 12. The resultant was stirred until dissolved and clarified, and allowed to stand and layer. An organic layer was obtained by separation, and was added with the washing solution. Under a controlled temperature of 40° C. to 45° C., the mixture was stirred for 60 min and allowed to stand and layer. An organic layer was obtained by separation, and was concentrated to dryness under reduced pressure, which was then dissolved by 120 mL of ethyl acetate. Under a controlled temperature of 50° C. to 60° C., hydrobromic acid was added dropwise to adjust pH=4±0.5 to form a salt, and the resultant was refluxed for 2 hours and cooled down to crystallize a crystal, which was then filtered and dried to obtain citalopram hydrobromide. Yield: 93%; purity: 99.8%; aldehyde impurity: 0.04%; impurity removal rate: 92.3%.

The above mentioned examples are only preferred examples of the present invention, and are not used to limit the invention. Any modification, equivalent replacement, improvement, etc. made within the spirit and principles of the invention, shall be included within the protection scope of the invention.

1. A purification method for citalopram or S-citalopram or salts thereof, comprising the following steps:
(a) treating a solution consisting of citalopram and a water-immiscible organic solvent with a washing solution, and carrying out a separation to obtain an organic layer containing citalopram; or, treating a solution consisting of S-citalopram and a water-immiscible organic solvent with a washing solution, and carrying out a separation to obtain an organic layer containing S-citalopram; and
(b) further separating the organic layer obtained in step (a) to obtain citalopram or S-citalopram; or further adding an acid to form a salt and carrying out a separation to obtain an acid salt of citalopram or an acid salt of S-citalopram;
wherein, the washing solution in step (a) is an aqueous solution comprising a washing agent, and the washing agent is selected from sulfite and thiosulfate.

2. The purification method according to claim 1, wherein the water-immiscible organic solvent in step (a) is selected from the group consisting of toluene, ethyl acetate, methyl isobutyl ketone, and chlorobenzene, or any combinations thereof.

3. The purification method according to claim 1, wherein the washing agent is selected from the group consisting of sodium dithionite, sodium thiosulfate, and sodium sulfite.

4. The purification method according to claim 1, wherein a mass ratio of the washing agent and water in the washing solution of step (a) ranges from 1% to 30%.

5. The purification method according to claim 1, wherein a molar ratio of the washing agent and citalopram or S-citalopram ranges from 0.05 to 1.5.

6. The purification method according to claim 1, wherein an inorganic base is added to the washing solution of step (a).

7. The purification method according to claim 6, wherein the inorganic base is selected from the group consisting of sodium carbonate, sodium bicarbonate, potassium carbonate, potassium bicarbonate, sodium hydroxide and potassium hydroxide, or any combinations thereof.

8. The purification method according to claim 6, wherein a mass ratio of the inorganic base and the washing agent ranges from 5% to 30%.

9. The method according to claim 1, wherein the acid in step (b) is selected from the group consisting of hydrochloric acid, hydrobromic acid, phosphoric acid, oxalic acid, fumaric acid, acetic acid, p-toluenesulfonic acid and methanesulfonic acid.

10. The purification method according to claim 1, wherein the solution consisting of citalopram and the water-immiscible organic solvent or the solution consisting of S-citalopram and the water-immiscible organic solvent in step (a) is obtained by: mixing citalopram hydrobromide or S-citalopram oxalate, water, and the water-immiscible organic solvent, adding a base to obtain a free base of citalopram or S-citalopram, and carrying out a separation to obtain an organic layer.

11. The purification method according to claim 1, wherein the washing agent is sodium dithionite.

12. The purification method according to claim 6, wherein the inorganic base is sodium bicarbonate.

13. The method according to claim 1, wherein the acid in step (b) is hydrobromic acid or oxalic acid.
